(19) United States
(12) Patent Application Publication
(10) Pub. No.: US 2004/0024352 A1
(43) Pub. Date: Feb. 5, 2004

(54) Breast pump system

(75) Inventors: Charles J. Renz, Briarcliff Manor, NY (US); David Robson, Riverside, RI (US); Marco Wo, Providence, RI (US); Christopher Kampf, Cranston, RI (US); Aidan J. Petrie, Jamestown, RI (US); Michael Pereira, Smithfield, RI (US)

Correspondence Address: CHARLES N.J. RUGGIERO, ESQ., OHLANDT, GREELEY, RUGGIERO & PERLE, L.L.P., 10th FLOOR, ONE LANDMARK SQUARE, STAMFORD, CT, 06901-2682, US

(73) Assignee: Playtex Products, Inc.

(21) Appl. No.: 10/453,261

(22) Filed: Jun. 3, 2003

Related U.S. Application Data
(63) Continuation-in-part of application No. 10/331,182, filed on Dec. 27, 2002.
(60) Provisional application No. 60/343,769, filed on Dec. 27, 2001.
(60) Provisional application No. 60/403,415, filed on Aug. 14, 2002.
(60) Provisional application No. 60/428,463, filed on Nov. 22, 2002.

(51) Int. Cl. A61M 1/06

(52) U.S. Cl. 604/74

(57) ABSTRACT

A breast pump system for obtaining breast milk is provided. The system sealingly separates the air flow from the breast milk and uses a single air tube for both positive pressure and negative pressure to be applied to a woman's breast. The breast pump can have a piston/cylinder device for generating pressure that allows a user to control suction and cycle time.

### RELATED APPLICATION

This application is a continuation-in-part of copending U.S. application Ser. No. 10/331,182 filed Dec. 27, 2002, which claims priority in U.S. Provisional Application Ser. No. 60/343,769, filed Dec. 27, 2001, U.S. Provisional Application Ser. No. 60/403,415, filed Aug. 14, 2002 and U.S. Provisional Application Ser. No. 60/428,463, filed Nov. 22, 2002, the disclosures of which are incorporated herein by reference.

### BACKGROUND OF THE INVENTION

1. Field of the Invention

The present invention relates to apparatus and methods for obtaining breast milk. More particularly, the present invention relates to a breast pump system that applies both a positive pressure and a negative pressure to a breast to express breast milk.

2. Description of the Related Art

Breast pump systems for obtaining breast milk, both manually and automatically, are known in the art. Conventional systems use a vacuum source to generate a negative pressure or vacuum that is transmitted through tubing to a breast hood or cup that is placed on the breast. This conventional device and method uses a negative pressure on the breast to express the breast milk. Such systems suffer from the drawback of applying only a vacuum source as negative pressure to the breast to induce the expression of breast milk.

### SUMMARY OF THE INVENTION

It is an object of the present invention to provide a breast pump system for expressing milk that applies both a positive pressure and a negative pressure to a breast to express the milk.

It is another object of the present invention to provide such a system that supplies the positive and negative pressure from a single source.

It is still another object of the present invention to provide such a system that facilitates control of the positive and negative pressure applied to the breast.

These and other objects and advantages of the present invention are provided by a breast pump system having a pressure source for generating a positive pressure and a negative pressure, and a breast cup in fluid communication with the pressure source, wherein the breast cup applies positive pressure and negative pressure to the breast.

The present invention also includes a breast pump system having a cylinder with a cylinder volume, a piston movably disposed in the cylinder, a motor operably connected to the piston to generate a pressure in the cylinder volume, and a breast cup in fluid communication with the cylinder volume, wherein the breast cup applies the pressure to the breast.

The present invention further includes a breast pump having a pressure source for generating a positive pressure and a negative pressure, and a controller operably connected to the pressure source, wherein the controller adjusts the positive and negative pressure and adjusts a cycle time between application of the positive and negative pressure to the breast.

The present invention additionally includes a breast pump having a pressure source for generating a pressure, and a controller operably connected to the pressure source, wherein the controller adjusts the pressure and adjusts a cycle time between application of the pressure to the breast. The controller also generates a wave signal in response to the pressure and the cycle time, and controls the pressure source in response to the wave signal.

The present invention includes a drive system for an expandable volume of a breast pump. The drive system has a motor having a first rotary output; a first gear system operably connected to the motor; and a second gear system operably connected to the first gear system and the expandable volume. The first gear system can have at least one belt. The second gear system has a rack gear and a pinion gear operably connected to the rack gear. The first gear system adjusts the rotary output provided to the second gear system to a second rotary output. The second gear system translates the second rotary output to a linear output.

The present invention includes a breast pump for supplying a pressure to a breast cup. The breast pump has an expandable volume in fluid communication with the breast cup to supply the pressure; a motor having a first rotary output; a first gear system operably connected to the motor; and a second gear system operably connected to the first gear system and the expandable volume. The first gear system can have at least one belt. The second gear system has a rack gear and a pinion gear operably connected to the rack gear. The first gear system adjusts the rotary output provided to the second gear system to a second rotary output. The second gear system translates the second rotary output to a linear output.

The system can also have a channel and the breast cup can have an air orifice, wherein the channel is connected to the air orifice and the pressure source, and the pressure source supplies reciprocating air flow through the channel between the breast cup and the pressure source. The channel can be flexible tubing. The pressure source can be a piston movably disposed in a cylinder. There can be a motor, a rack having first teeth and a gear having second teeth. The rack is preferably connected to the piston, the gear is preferably operably connected to the motor, and the first teeth engage with the second teeth to reciprocally move the piston in the cylinder.

The piston can have a sealing member disposed between the piston and the cylinder. The sealing member can be an o-ring disposed on the piston. The piston can have a substantially cylindrical shape with a circumferential wall, and the sealing member can be a plurality of gaskets disposed on the circumferential wall. The piston can have a substantially cylindrical shape with a circumferential wall having a circumferential channel formed therein, and wherein the sealing member is at least partially disposed in the channel. The piston can have a v-shaped cross section with a leading edge and a trailing edge, and wherein the leading edge and the trailing edge form a sealing engagement with the cylinder.

The piston can be flexibly secured to the rack. The piston can have a recess and the rack can have a first end with an abutment formed therein, wherein the abutment is flexibly secured in the recess. The recess and the first end can have detent structures. The cylinder can have a first diameter and an air hole, wherein the air hole has a second diameter and is in fluid communication with the atmosphere, and wherein the first diameter is significantly larger than the second diameter.

There can also be a controller operably connected to the motor, wherein the motor is reversible and the controller reverses the motor based upon a positive or negative pressure limit. There can be a controller operably connected to the motor, wherein the motor is a reversible motor, wherein the controller determines a distance that the piston has traveled relative to the cylinder and wherein the controller reverses the motor based upon the distance. There can be a photo-sensor that generates a signal in response to the distance, wherein the signal is transmitted to the controller, and wherein the controller reverses the motor in response to the signal.

The rack can have a plurality of openings formed therein, wherein the photo-sensor is operably aligned with the openings, and wherein the signal is generated based upon a count of the openings moving past the photo-sensor. There can also be a position switch, wherein the photo-sensor is operably aligned with the position switch to generate a position signal, wherein the position signal is transmitted to the controller, and wherein the controller resets the count in response to the position signal. There can be a controller operably connected to the motor, wherein the motor has variable speed, and the controller adjusts the speed based upon a desired cycle time for applying the positive or negative pressure to the breast. The controller can have a user interface, the desired cycle time can be inputted into the user interface, and the desired cycle time can be transmitted to the controller from the user interface.

There can be a controller having a user interface and operably connected to the pressure source, wherein the controller adjusts the positive or negative pressure generated by the pressure source in response to a signal transmitted from the user interface. There can also be a controller having a user interface and operably connected to the pressure source, wherein the controller adjusts a cycle time for applying the positive or negative pressure to the breast in response to a signal transmitted to the controller from the user interface. There can be a controller that generates a wave signal in response to an amount of pressure and a cycle time between the positive and negative pressure, and controls the motor in response to the wave signal. There can be a user interface, wherein a desired wave signal is inputted into the user interface, the desired wave signal is transmitted to the controller from the user interface, and the controller adjusts the wave signal to correspond to the desired wave signal.

The cylinder can be in fluid communication with a pressure relief valve. The pressure relief valve can be adjustable. The pressure source can have a housing with a storage compartment formed therein, and wherein the flexible tubing is removably stored in the storage compartment. The housing can have an air outlet with a first end and a second end, wherein the first end is in fluid communication with the pressure source and the second end is disposed in the storage compartment.

There can be a t-connector having an inlet, a first outlet, a second outlet and a plug, wherein the inlet is in fluid communication with the first and second outlets, and wherein the plug is selectively sealingly engageable with the first outlet or the second outlet. The t-connector can have an outer surface and the plug is tethered to the outer surface. The controller can have a user interface, a desired level of the positive or negative pressure can be inputted into the user interface, and the controller can adjust the positive or negative pressure in response to a signal transmitted from the user interface.

The first gear system can have a first belt and a second belt. The first belt can be non-toothed and the second belt can be toothed. The first belt can be resilient. The first belt can be a plurality of belts. The first gear system can have a first pulley and a second pulley. The first pulley can have a first circumference and a first channel formed along the first circumference. The second pulley can have a second circumference and a plurality of teeth formed along the second circumference. The first belt can be partially disposed in the first channel and the second belt can engages the plurality of teeth of the second pulley.

The motor can have a drive shaft with an annular channel formed therein. The first belt can be partially disposed in the annular channel. The second pulley can be secured to the pinion gear. The expandable volume can be a cylinder and a piston movable in the cylinder, with the rack gear secured to the piston. The rack gear can be flexibly secured to the piston. The cylinder can have a first diameter and an air hole. The air hole can have a second diameter and be in fluid communication with the atmosphere. The first diameter can be significantly larger than the second diameter.

### BRIEF DESCRIPTION OF THE DRAWINGS

Other and further objects, advantages and features of the present invention will be understood by reference to the following:

FIG. 1 is a front perspective view of a breast pump of the breast pump system of the present invention;

FIG. 2 is a front perspective view of the breast pump of FIG. 1 in an opened position;

FIG. 3 is an exploded perspective view of the breast pump of FIG. 1;

FIG. 4 is a top view of the breast pump of FIG. 1 without the cover;

FIG. 5 is an exploded perspective view of a piston and cylinder of the present invention;

FIG. 6 is an exploded side view of a portion of the piston and cylinder of FIG. 5;

FIG. 7 is a front perspective view of the piston of FIG. 5;

FIG. 8 is an exploded perspective view of an alternative embodiment of the piston of the present invention;

FIG. 9 is an exploded perspective view of a pressure relief valve of the system of FIG. 1;

FIG. 10 is a cross-sectional plan view of the cylinder of FIG. 5;

FIG. 11 is a front perspective view of a breast cup of the present invention;

FIG. 12 is a side cross-sectional view of the breast cup of FIG. 11;

FIG. 13 is a rear perspective view of a T-connector of the present invention;

FIG. 14 is a flow chart depicting a method for pumping a breast according to the system of FIGS. 1 and 11;

FIG. 15 is a top perspective view of a preferred embodiment of breast pump for the breast pump system of the present invention;

FIG. 16 is a top view of the breast pump of FIG. 15;

FIG. 17 is a top perspective view of the drive system of the breast pump of FIG. 15;

FIG. 18 is a side perspective view of the drive system of FIG. 17;

FIG. 19 is a top perspective view of a portion of the gear reduction system of the drive system of FIG. 15, partially assembled;

FIG. 20 is a top perspective view of an alternative embodiment of breast pump for the breast pump system of the present invention;

FIG. 21 is a top view of the breast pump of FIG. 20;

FIG. 22 is a top perspective view of the drive system of the breast pump of FIG. 20;

FIG. 23 is a side perspective view of the drive system of FIG. 20;

FIG. 24 is a top perspective view of the motor of the drive system of FIG. 20;

FIG. 25 is a top perspective view of a portion of the gear reduction system of the drive system of FIG. 20, partially assembled; and

FIG. 26 is a top perspective view of the gear reduction system of the drive system of FIG. 20, partially assembled.

### DESCRIPTION OF THE INVENTION

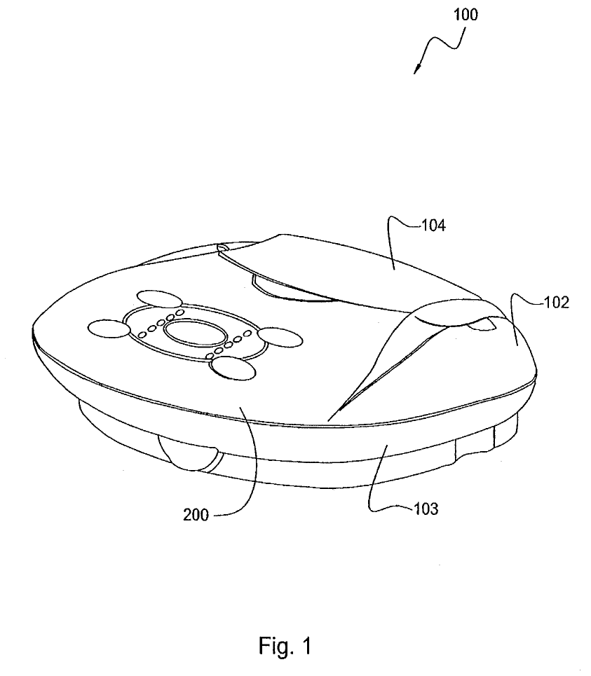

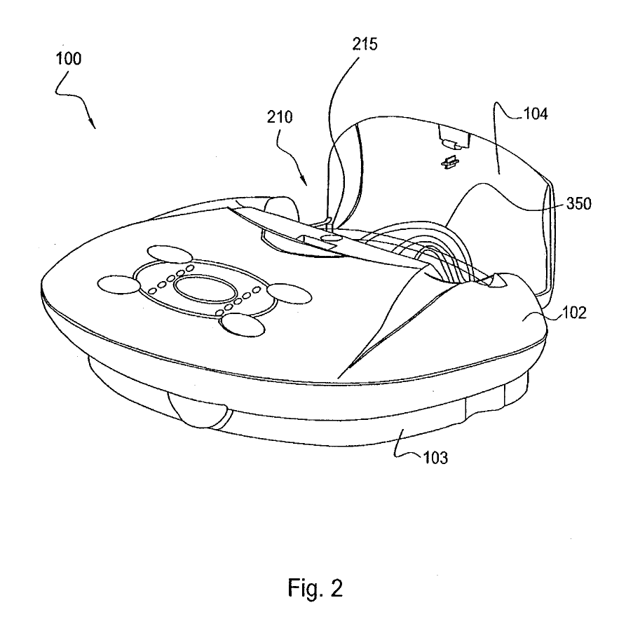

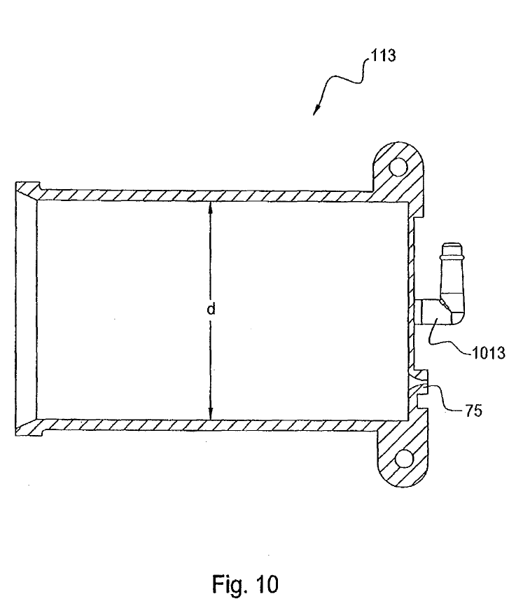

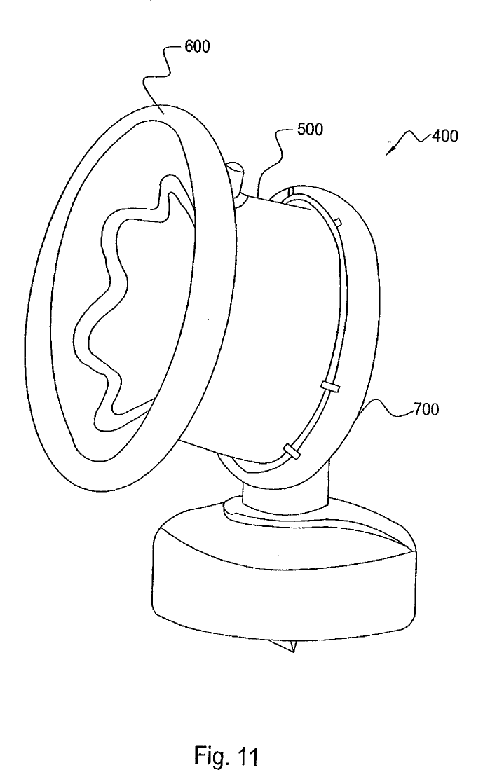

Referring to the drawings and, in particular, FIGS. 1 and 2, there is shown a breast pump of the present invention generally represented by reference numeral 100. Breast pump 100, along with breast cup 400 shown in FIG. 11, form the major components of the breast pump system of the present invention. Breast pump 100 has a top housing 102 and a bottom housing 103 that are adapted to form an assembled unit.

Referring to FIGS. 1 through 3, top housing 102 has a substantially ellipsoidal shape with a flat front face 200 and a storage compartment 210 having a compartment door 104. Preferably, door 104 is hingedly connected to top housing 102 to form a selectively sealable storage compartment 210 for storing air tubing or conduit 350 that connects breast pump 100 to the other components of the system, which will be discussed later in greater detail.

Face 200 can receive a button pad 105 having an LED cover 106. Pad 105 is used by the consumer to control breast pump 100. Bottom housing 103 can securely house the various components of the breast pump, which include a rack gear 109, a pinion gear 110 that can engage the rack gear, a piston 112, a cylinder 113 that can receive the piston, and a motor 125 having a shaft 126 upon which the pinion gear is mounted. Due to this design, breast pump 100 provides pumping with low noise. Breast pump 100 can be made of any rigid material, such as, for example, plastic.

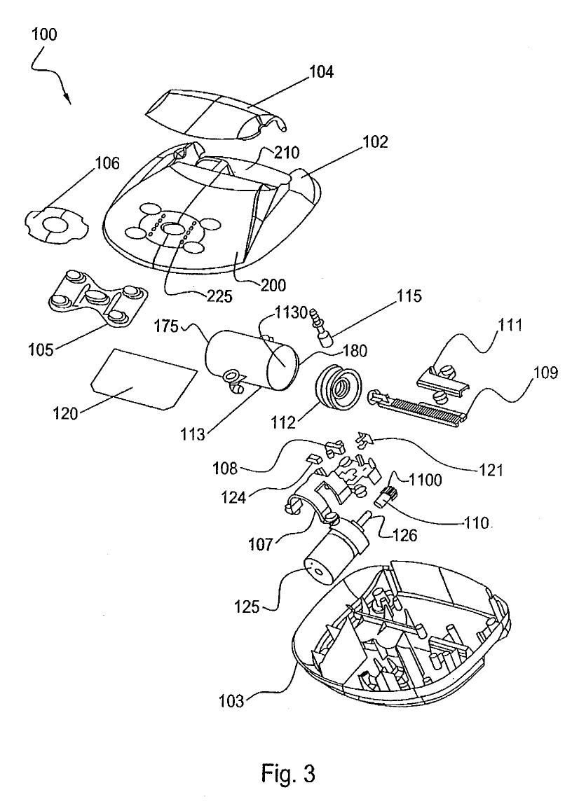

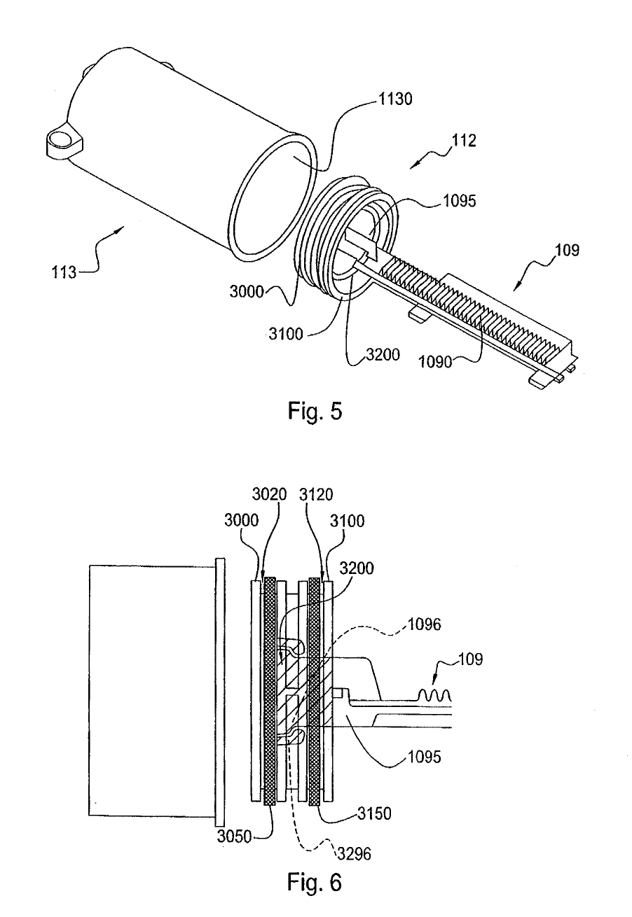

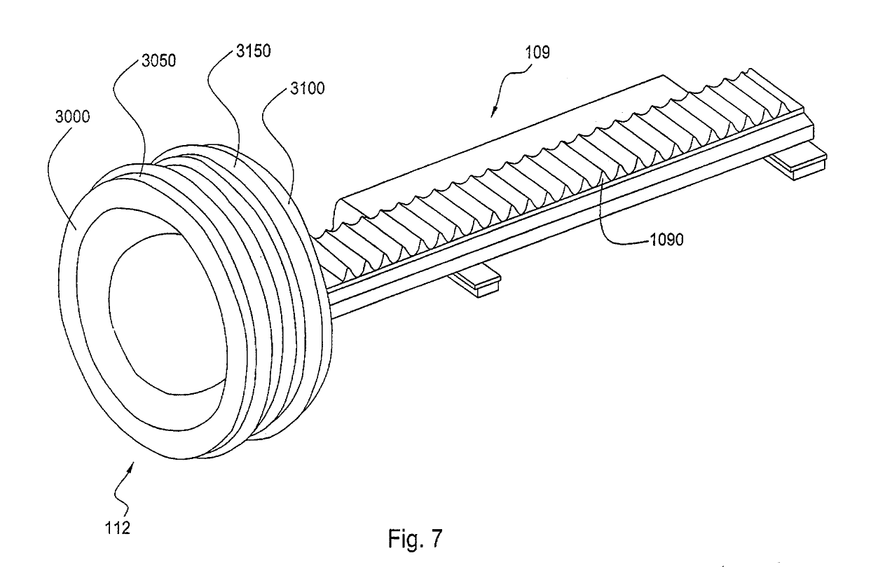

Referring to FIGS. 3 through 7, breast pump 100 utilizes piston 112 and cylinder 113 to create both a positive pressure and a negative pressure for obtaining breast milk. Piston 112 is driven by rack gear 109, which is affixed thereto. Piston 112 has a substantially cylindrical-shape with a first head 3000 and a second head 3100. First and second heads 3000, 3100 preferably have annular channels 3020, 3120 formed therein, respectively. Channels 3020, 3120 are disposed along the outer circumference of first and second heads 3000, 3100, respectively. Preferably, channels 3020, 3120 are centrally located along the outer circumference of first head 3000 and second head 3100. Seated in channels 3020, 3120 are sealing members 3050, 3150, respectively. Preferably, sealing members 3050, 3150 are o-ring gaskets. Sealing members 3050, 3150 have a diameter or width that is larger than the depth or height of channel 3020 and channel 3120. Sealing members 3050, 3150 extend beyond the outer circumference of first head 3000 and second head 3100 forming a sealing engagement with an inner surface 1130 of cylinder 113 as piston 112 is driven back and forth in the cylinder.

The use of multiple sealing members, i.e., o-ring gasket 3050 and o-ring gasket 3150 on piston 112, provide a double sealing to increase the efficiency of creating the positive pressure and negative pressure. While this embodiment uses two sealing members to create two separate sealing surfaces, any number of sealing members can be used to create any number of sealing surfaces for sealing piston 112 with cylinder 113. Additionally, while this embodiment uses piston 112 having o-ring sealing gaskets 3050, 3150, alternative sealing structures can be used between the piston and cylinder 113.

Rack gear 109 has teeth 1090 that engage with pinion gear 110 having teeth 1100. Pinion gear 110 is operatively connected to motor 125, preferably via shaft 126. When motor 125 is activated, shaft 126 and pinion gear 110 rotate. Teeth 1090 on rack 109 and teeth 1100 on pinion 110 mesh and translate the reciprocal rotational motion of motor 125 and shaft 126 into a reciprocal longitudinal motion along a single axis in both directions.

Preferably, rack gear 109 has a first end 1095 that engages with a recess 3200 formed in piston 112. Recess 3200 is preferably centrally located in piston 112. First end 1095 of rack gear 109 preferably has a snap fit or friction fit engagement with recess 3200 of piston 112. Preferably, there are detent structures 1096, 3296 formed on first end 1095 and recess 3200, respectively. This facilitates production of these components and also provides for any slight pivotal movement that may be required of piston 112 with respect to rack gear 109.

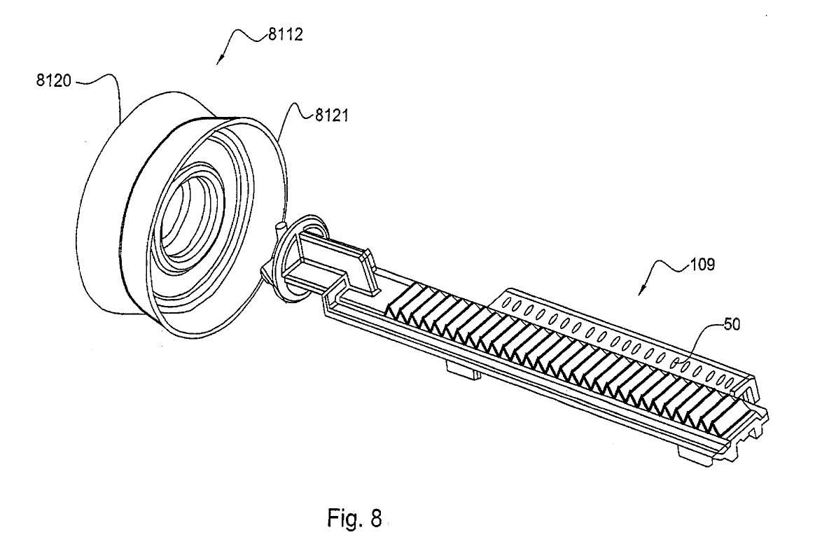

An alternative embodiment of a piston is shown in FIG. 8 and generally represented by reference numeral 8112. Piston 8112 has a substantially V-shape with a leading edge 8120 and a trailing edge 8121. Leading edge 8120 and trailing edge 8121 sealingly engage an inner surface 1130 of cylinder 113 as piston 8112 is driven back and forth in the cylinder. The use of multiple edges, i.e., leading edge 8120 and following edge 8121, on piston 8112 that sealingly engage inner surface 1130 of cylinder 113, provide a double sealing to increase the efficiency of creating the positive pressure and negative pressure.

Referring to FIGS. 3 through 7, motor 125 is preferably variable speed. This allows a user to control and vary the cycle time of the pumping of the breast. Breast pump 100 further has a motor cover 107 and a bearing 108 to reduce vibration and to secure motor 125 to bottom housing 103.

The positive and negative pressures can be varied by changing the displacement of air volume in cylinder 113. In this embodiment, this is done by use of a photoelectric or photo-sensor system. The photo-sensor system has two or more photo-sensors 121 and a position switch 124. The photo-sensors 121 count the number of openings 50 on rack gear 109, as the rack gear moves back and forth. Thus, a user can control the distance that rack gear 109 travels and correspondingly control the air volume displacement in cylinder 113.

To ensure that piston 112 is properly moving to the front of cylinder 113, the photo-sensor system further includes position switch 124, preferably located at the front of the cylinder, which acts as a starter for the counter. Alternatively, the position switch can be an opening 50 having a different size or shape that is detectable by photo-sensor 121.

Rack gear 109 can also have a safety mechanism attached thereto. Photo-sensor 121 will be reading openings 50 as rack gear 109 moves backwards. If for some reason rack gear 109 misses its target and moves too far, the safety will trigger the position switch. When the position switch is triggered while rack gear 109 is moving backwards, the software can trigger the system to move forward again and return to the position position.

Breast pump 100 has a guide cover 111 positioned over rack gear 109. Guide cover 111 provides added stability to the breast pump by guiding and vibration dampening the reciprocal movement of rack gear 109. Guide cover 111 also provides accuracy to the photo-sensor system by reducing the risk of misalignment of photo-sensors 121 and openings 50.

The photo-sensor system and motor 125 are preferably connected to a PC or circuit board 120. Thus, the distance piston 112 travels, which translates to the amount of positive and negative pressure, and the piston speed, which translates to the cycle time, are electronically controlled.

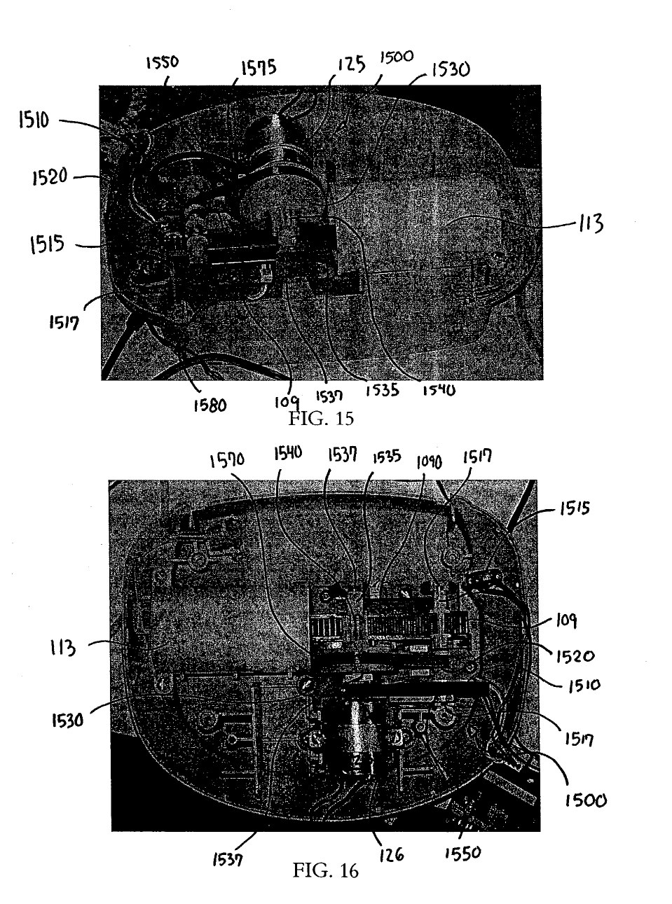

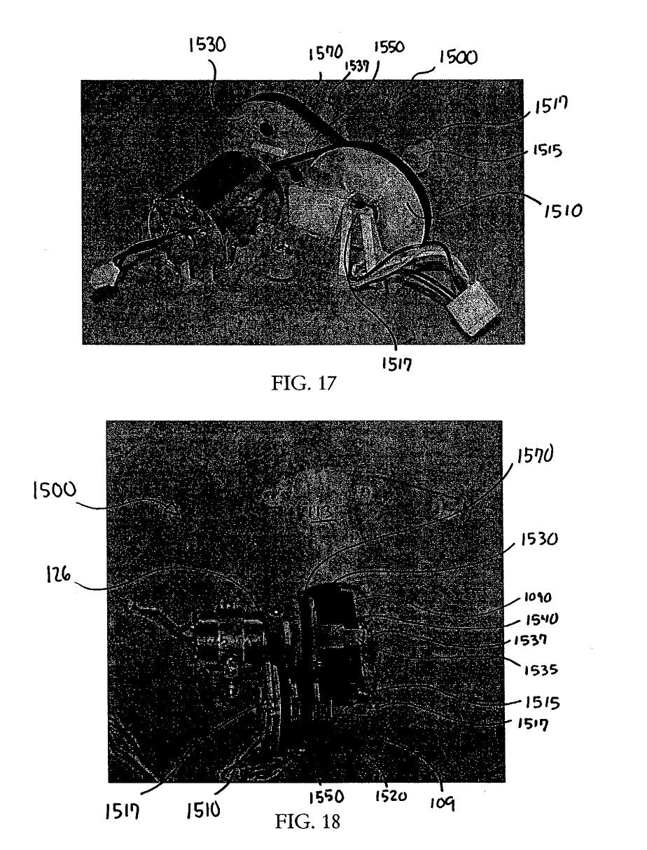

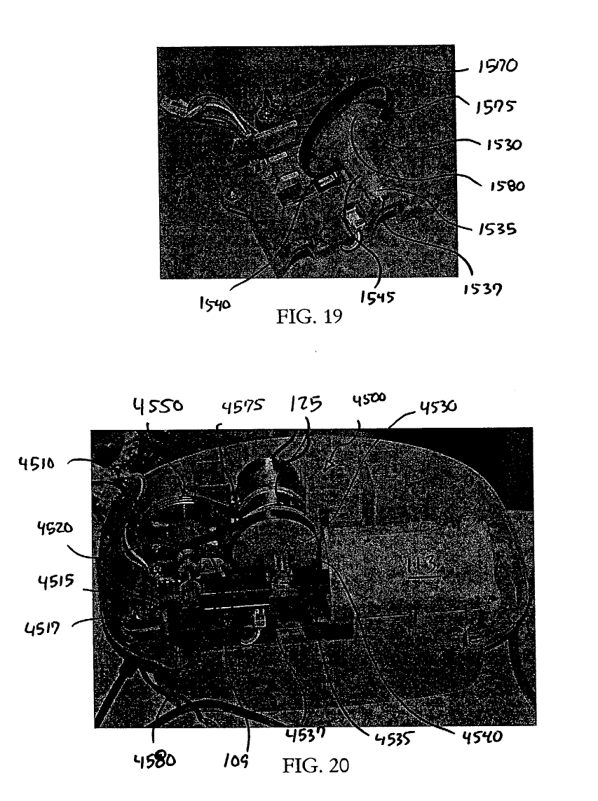

Referring to FIGS. 15 through 19, a preferred embodiment of a drive system of the present invention is shown and generally represented by reference numeral 1500. Drive system 1500 is usable with breast pump 100 of FIGS. 1 through 7 to provide the linear reciprocal movement of piston 112 with cylinder 113.

Drive system 1500 is a belt drive system for a rack and pinion drive having gear reduction incorporated therein. Drive system 1500 has a first drive wheel or pulley 1510; a second gear, drive wheel or pulley 1520 secured to the first drive wheel 1510; a third gear, drive wheel or pulley 4530; and a pinion gear 1540 secured to the third gear.

First drive wheel 1510 is operably connected to motor drive shaft 126 by a first belt 1550. In the preferred embodiment, first belt 1550 is a non-toothed belt. More preferably, first belt 1550 has resiliency or flexibility. The use of flexible or resilient belt 1550 provides a secure connection between drive shaft 126 and first drive wheel 1510 and also reduces noise and vibration. Drive shaft 126 and first drive wheel 1510 have smooth outer surfaces upon which the first belt 1550 is secured.

First drive wheel 1510 is operably connected to second gear 1520 by a first co-axial shaft 1515. In the preferred embodiment, first shaft 1515 is rotatably mounted between opposing first bearings 1517. However, alternative rotatable mounting arrangements or securing structures could also be used. To reduce noise and vibration, motor shaft 126 and first drive wheel 1510 are made of metal. First drive wheel 1510 and second gear 1520 have different diameters that partially provide for gear reduction between motor shaft 126 and pinion gear 1540.

Second gear 1520 is operably connected to third gear 1530 by a second belt 1570. Preferably, second belt 1570 has teeth 1575 that mesh with teeth 1580 formed along the circumference of second gear 1520 and third gear 1530. Second and third gears 1520, 1530 have different diameters that partially provide for gear reduction between motor shaft 126 and pinion gear 1540. Drive system 1500 can also have a tension pulley 1580 that provides tension to second belt 1570.

Third gear 1530 is operably connected to pinion gear 1540 by a second co-axial shaft 1535. In the preferred embodiment, second shaft 1535 is rotatably mounted between opposing second bearings 1537. However, alternative rotatable mounting arrangements or securing structures could also be used. Preferably, third gear 1530 is integrally molded with pinion gear 1540 along second shaft 1535.

Pinion gear 1540 has teeth 1545 that engage with teeth 1090 of rack gear 109. When motor 125 is activated, the rotational motion of shaft 126 is translated into a reciprocal longitudinal motion along a single axis of rack gear 109 in both directions. Drive system 1500, through use of first and second belts 1550, 1570 and first, second and third drive wheels or gears 1510, 1520, 1530, is able to provide a desired ratio of movement between motor shaft 126 and pinion gear 1540, i.e., gear reduction.

The use of a combination of the non-toothed belt 1550 and the toothed belt 1570 reduces noise and vibration, while maintaining a secure, sturdy drive system 1500 that is able to provide the necessary back and forth linear motion at the desired speeds and pressure for breast pump 100.

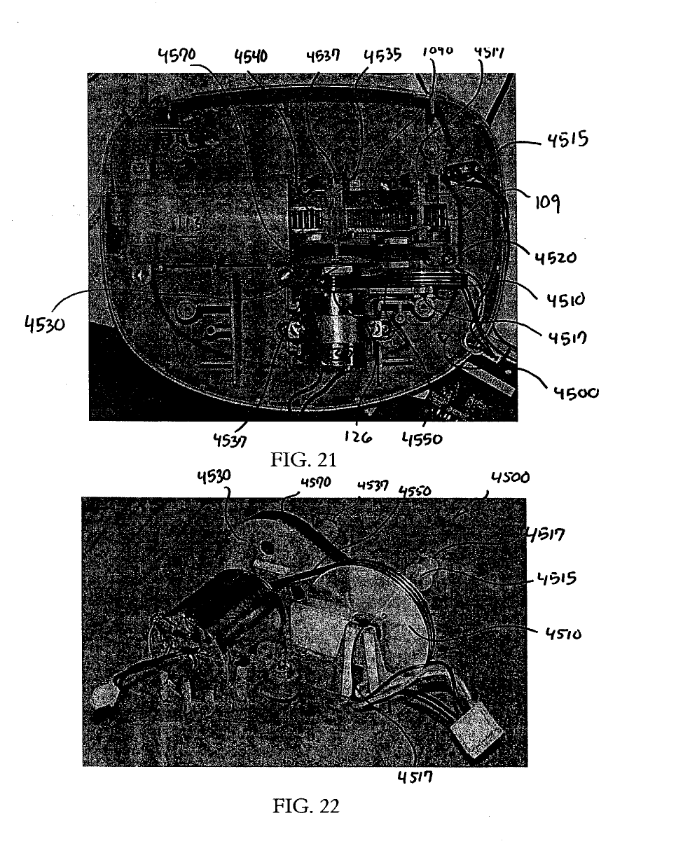

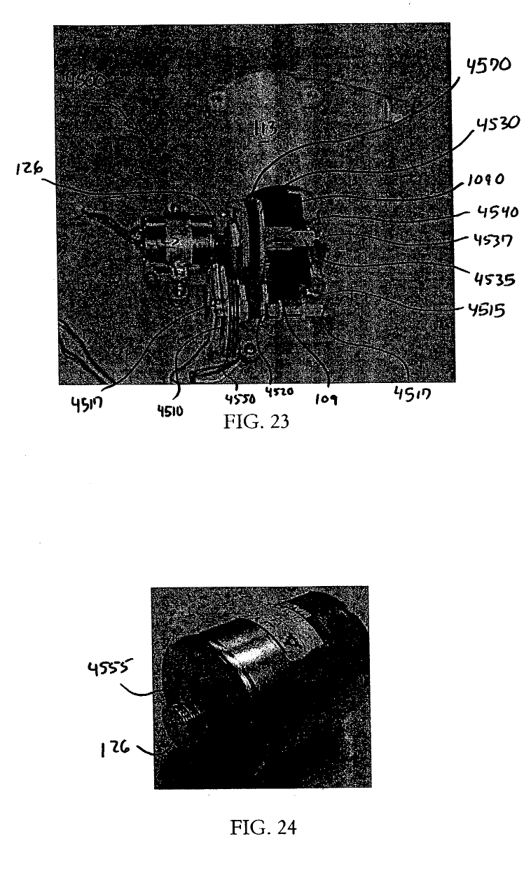

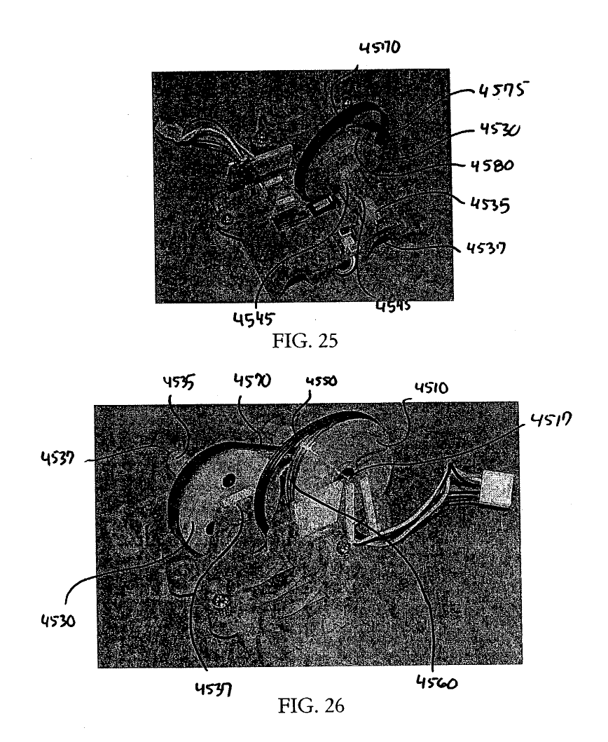

Referring to FIGS. 20 through 26, an alternative embodiment of a drive system of the present invention is shown and generally represented by reference numeral 4500. Drive system 4500 is also usable with breast pump 100 of FIGS. 1 through 7 to provide the linear reciprocal movement of piston 112 with cylinder 113.

Drive system 4500 is a belt drive system having gear reduction incorporated therein. Drive system 4500 has a first gear, drive wheel or pulley 4510; a second gear, drive wheel or pulley 4520 secured to the first gear; a third gear, drive wheel or pulley 4530; and a pinion gear 4540 secured to the third gear.

First gear 4510 is operably connected to motor drive shaft 126 by a first belt 4550. In the preferred embodiment, first belt 4550 is a plurality of belts, and more preferably, three belts. First belts 4550 are preferably non-toothed belts. More preferably, first belts 4550 are o-rings having resiliency or flexibility. The use of flexible or resilient belts 4550, such as, for example, o-rings, provides a secure connection between drive shaft 126 and first gear 4510, and also reduces noise and vibration. Drive shaft 126 and first gear 4510 have annular channels 4555, 4560, formed therein, respectively. Annular channels 4555, 4560 are guides that assist in holding first belts 4550 in place and facilitate assembly of drive system 4500.

First gear 4510 is operably connected to second gear 4520 by a first co-axial shaft 4515. In this alternative embodiment, first shaft 4515 is rotatably mounted between opposing first bearings 4517. However, alternative rotatable mounting arrangements or securing structures could also be used. To reduce noise and vibration, motor shaft 126 and first gear 4510 are made of metal. First and second gears 4510, 4520 have different diameters that partially provide for gear reduction between motor shaft 126 and pinion gear 4540.

Second gear 4520 is operably connected to third gear 4530 by a second belt 4570. Preferably, second belt 4570 has teeth 4575 that mesh with teeth 4580 formed along the circumference of second gear 4520 and third gear 4530. Second and third gears 4520, 4530 have different diameters that partially provide for gear reduction between motor shaft 126 and pinion gear 4540. Drive system 4500 can also have a tension pulley 4580 that provides tension to second belt 4570.

Third gear 4530 is operably connected to pinion gear 4540 by a second co-axial shaft 4535. In this alternative embodiment, second shaft 4535 is rotatably mounted between opposing second bearings 4537. However, alternative rotatable mounting arrangements or securing structures could also be used. Preferably, third gear 4530 is integrally molded with pinion gear 4540 along second shaft 4535.

Pinion gear 4540 has teeth 4545 that engage with teeth 1090 of rack gear 109. When motor 125 is activated, the rotational motion of shaft 126 is translated into a reciprocal longitudinal motion along a single axis of rack gear 109 in both directions. Drive system 4500, through use of first and second belts 4550, 4570 and first, second and third gears 4510, 4520, 4530, is able to provide a desired ratio of movement between motor shaft 126 and pinion gear 4540, i.e., gear reduction.

The use of a combination of the non-toothed o-ring belts 4550 and the toothed belt 4570 reduces noise and vibration, while maintaining a secure, sturdy drive system 4500 that is able to provide the necessary back and forth linear motion at the desired speeds and pressure for breast pump 100.

The embodiments of the drive systems 1500 and 4500 described above utilize belts for gear reduction. However, alternative embodiments can use a gear-box that reduces the gearing to the desired ratio that is transferred to the rack and pinion gearing that drives breast pump 100.

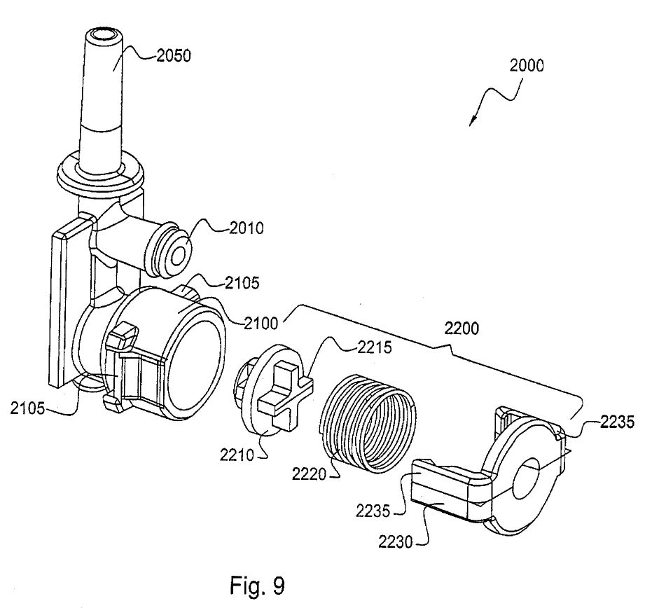

Referring back to FIGS. 3 through 9, cylinder 113 has a supply tube 116 that is secured to a supply connector 115 for supplying the positive and negative pressure to breast cup 400. Preferably, supply connector has an outlet 215 disposed in storage compartment 210. Air tubing 350 can be secured to outlet 215 and also secured to breast cup 400. Storage compartment 210 can be opened or closed during the pumping operation. Cylinder 113 is in fluid communication with a pressure relief valve 2000 (shown in FIG. 9) that is preferably set at about 1.5 in. Hg.

Pressure relief valve 2000 has an intake 2010 and an exhaust 2050. Intake 2010 is in fluid communication with cylinder 113 and exhaust 2050 is in fluid communication with breast cup 400, by tubing 350. Pressure relief valve 2000 has a relief exhaust 2100 that is in fluid communication with intake 2010 and exhaust 2050. Relief exhaust 2100 is substantially tubular and is secured to a relief assembly 2200.

Relief assembly 2200 has a flexible insert 2210, a biasing member 2220 and a retaining member 2230. Flexible insert 2210 sealing engages with the inner surface of relief exhaust 2100 to prevent air from exiting through the relief exhaust. Insert 2210 has a securing member 2215 that mates with biasing member 2200. In this embodiment, securing member 2215 is a cross-shaped structure that is received in the inner volume of biasing member 2200. Preferably, biasing member 2200 is a spring. More preferably, biasing member 2200 is a coil spring. Retaining member 2230 is a cap-like structure having opposing retaining arms 2235 that engage with a corresponding pair of engaging protrusions 2105 positioned on the outer surface of relief exhaust 2100. Insert 2210 and spring 2220 are held in the inner volume of relief exhaust 2100 by cap 2230.

Spring 2220 has a biasing strength or resistance that is equal to the relief pressure of relief pressure valve 2000. When a positive pressure exceeds the relief pressure, which in this embodiment is preferably set at about 1.5 in. Hg, the force created on the inner surface of insert 2210 overcomes the biasing force of spring 2220 and the insert moves toward cap 2230 and outside of the inner volume of relief exhaust 2100. Air exits pressure relief valve 2000 through relief exhaust 2100 until the positive pressure in the pressure relief valve decreases below the biasing strength of spring 2220, at which time insert 2210 moves back in the inner volume of the relief exhaust, sealingly engaging the inner surfaces of the relief exhaust.

Alternatively, the pressure relief valve can be made adjustable so that the “massage strength”, i.e., the amount of positive pressure on the user's breast, can be controlled. Circuit board 120, shown in FIG. 3, allows a user to program several levels of speed and several levels of suction.

In this embodiment, the speed (cycle time) ranges from about 45 cycles/minute (cpm) to about 75 cpm. The present invention provides for pre-set programming of a number of speed levels within the speed range. Preferably, the number of levels can be from about two to about eight levels. More preferably, the user can program five levels of speeds within the speed range. The present invention also envisions programming of the speed levels by the user.

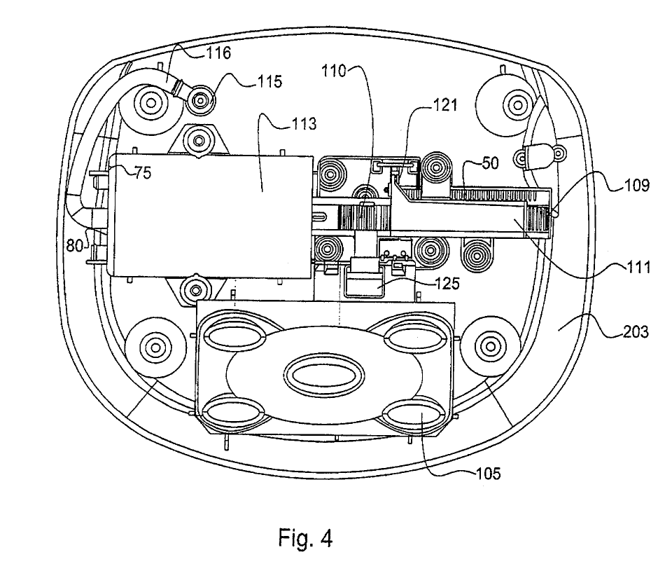

The suction range for use with a single breast cup 400 and the preferred drive system 1500 shown in FIGS. 15 through 21, is from about 3 in. Hg to about 10 in. Hg, and from about 3 in. Hg to about 8 in. Hg for two breast cups. The suction range for use with a single breast cup 400 and the gear box system shown in FIGS. 3 and 4 is from about 3 in. Hg to about 9 in. Hg, and from about 3 in. Hg to about 8 in. Hg for two breast cups. The present invention provides for pre-set programming of a number of suction levels within the suction range. Preferably, the number of levels can be from about two to about eight levels. More preferably, the user can program five levels of suction within the suction range. The present invention also envisions programming of the suction levels by the user.

Computer software can also be used to control the amount of positive and negative pressure. This allows the amounts of positive and negative pressure to be personalized for the user and also varied over the duration of the pumping process to maximize efficiency.

Breast pump 100 is preferably controlled by a software-driven circuit board 120, along with a gear motor 125, a rack and pinion set 109, 110, and a piston system 112, 113. The software and system are designed to provide maximum flexibility and to facilitate changing of the pressure curve or “wave.” This is feasible because the software controls the speed of motor 120 and the distance that piston 112 will travel in cylinder 113. The distance piston 112 travels relates to the pressure levels. By controlling speed and pressure levels with software, the pressure curve or “wave” can be controlled.

Once a determination is made that there is a specific “wave” or pressure curve that is similar to the sucking of an infant or most comfortable to the mother, then the desired wave can be obtained by changing the timing (motor speed and piston distance). Through use of software, a user has the ability to apply memory to a particular pressure curve and the variation of that pressure curve over time so as to maximize the comfort for the user.

In this embodiment, a sine wave is used for the control of breast pump 100. This is based on the assumption that the most comfortable pressure curve would be one that increases and decreases in pressure gradually, similar to a sine wave, without sharp pressure peaks and valleys providing a pinching feeling on the user. The back and forth motion of piston 112 approximates the desired sine wave. However, to avoid sharp pressure peaks, the timing of piston 112 is slowed down at these peaks, and the pressure is held constant for a duration of time at the maximum and minimum suction points on the wave. This results in a pressure curve having a steady sine wave that is more comfortable to the user.

Alternative waves can also be used for the pressure curve if such a wave is determined to be desired by the mother. For example, if a mother prefers a “saw tooth” pressure curve with sharp peaks, the timing of piston 112 can be changed to simply cycle back and forth, minimizing the pause when piston 112 changes direction. Also, for example, if a mother prefers a “square curve”, the timing of piston 112 can be changed to hold the piston position when the piston is ready to change direction, and then quickly ramp down and hold its position again before it ramps back up. This will create a “square curve” wave.

Use of software control provides for numerous choices of waves or pressure curves. This further allows the flexibility to change or offer greater choice with one breast pump 100. In contrast, contemporary pumps have the drawback of not allowing the flexibility of changing pressure curve waves.

Cylinder 113 has a pressure differential hole 75. Preferably, pressure differential hole 75 is located along bottom face 80 of cylinder 113. Pressure differential hole 75 is substantially smaller than exhaust hole 1013 and supply tube 116 through which the air flows for generating the positive and negative pressure. Pressure differential hole 75 provides a variance in the amount of positive pressure as compared to the amount of negative pressure. Pressure differential hole 75 is effective for the higher ranges of vacuum to provide the “lost” air at the end of the vacuum stroke. On the positive pressure stroke, a small amount of air will be released through pressure differential hole 75 but the air will be reintroduced during the negative pressure stroke when the level of pressure is higher.

Referring to FIG. 10, cylinder 113 is formed as a zero-draft cylinder. The outer diameter of piston 112 creates a seal with the inner diameter d of cylinder 113 to move the volume of air inside the cylinder, creating vacuum and pressure on the breast. Breast pump 100 requires a cylinder 113 that has a consistent inner diameter d through the entire length of the cylinder to create an appropriate seal while minimizing interference or resistance to piston 112. Typical injection molded parts require a draft angle that would create a non-uniform inner diameter d of cylinder 113.

Cylinder 113 is preferably molded as a zero-draft cylinder that provides a uniform inner diameter d and more preferably, molded in a single piece. As shown in FIG. 10, cylinder 113 is a one piece, plastic injection molded part. A two-part cylinder or a machined-cylinder have drawbacks which the single piece, zero draft cylinder 133 overcomes. The two-part cylinder requires an extruded tube attached to an end cap, with the two parts joined using a weld or using an adhesive. The machined part is typically a metal tube. One of the advantages to the zero-draft, one-piece cylinder 113 is that it is injection moldable.

Referring to FIGS. 3 through 10, button pad 105 is the user interface or control mechanism for breast pump 100. Button pad 105 has a pair of positive and negative keys for increasing or decreasing the level of suction and speed. Pad 105 further includes an on/off switch.

Due to the reciprocal back and forth motion of piston 112 in cylinder 113, breast pump 100 supplies both a positive pressure and a negative pressure to a woman's breast through a single hose or tubing 350. While this embodiment uses a piston/cylinder mechanism to create positive and negative pressure, alternative expandable volumes or pressure sources can also be used. Such alternative embodiments include a bellows mechanism or a diaphragm that would require fewer parts.

Referring to FIGS. 11 and 12, Breast Cup 400 of the present invention is shown. An example of breast cup 400 is disclosed in the co-pending and commonly owned U.S. application Ser. No. 10/331,183, filed Dec. 27, 2002, the disclosure of which is incorporated herein by reference. Breast cup 400 has a housing 500 having an air orifice 560, a flexible insert 600, and a holder 700. Housing 500 is a rigid structure and flexible insert 600 is a flexible structure. Housing 500 is adapted for sealing engagement with insert 600 to form a displacement volume 510 between the housing and the insert. Air orifice 560 is in fluid communication with displacement volume 510.

Breast pump 100 is placed in fluid communication with breast cup 400 via air tubing 350 that is connected to air orifice 560 and in fluid communication with cylinder 113. Breast pump supplies both a positive and negative pressure to breast cup 400. The positive and negative pressure created by breast pump 100 causes air to flow through air orifice 560 into and out of displacement volume 510. The positive and negative pressure supplied to breast cup 400 causes flexible insert 600 and, in particular, displacement volume 510 to expand and contract to apply reciprocating positive and negative forces on the user's breast.

Breast pump 100 and breast cup 400 are able to apply both a positive and a negative pressure to a user's breast through a single air tubing 350, which is connected to air orifice 560.

The volume disposed in displacement volume 510 is preferably between 22 to 52 cubic centimeters, and more preferably between 32 to 42 cubic centimeters. The expandable and contractible displacement volume 510 provides an upper limit to the amount of negative pressure that can be applied to a user's breast, which can further serve as a safety feature in use of breast pump 100. Additionally, the sealing engagement of insert 600 and housing 500 provides a barrier between the user's breast and breast pump 100 to prevent any breast milk from entering air tubing 350 or the breast pump.

While the preferred embodiment of the breast pump system uses breast cup 400 having a displacement volume 510 in fluid isolation from the user's breast, alternative breast cups can also be used with breast pump 100. The unique features of the breast pump system of the present invention can be used with other types of breast cups, such as, for example, the control system of the present invention or the rack and pinion driving mechanism.

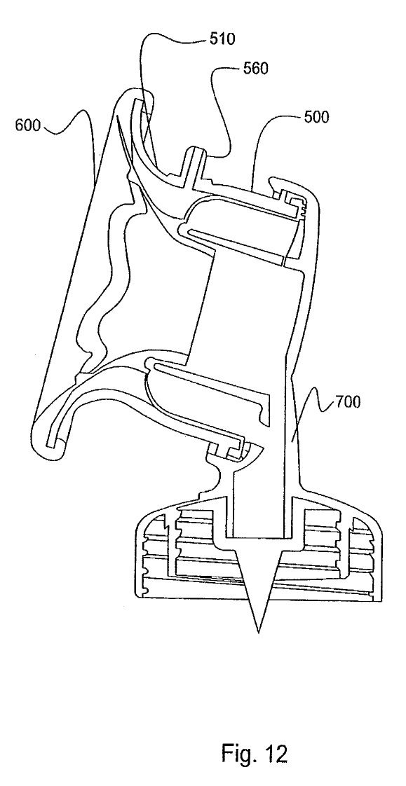

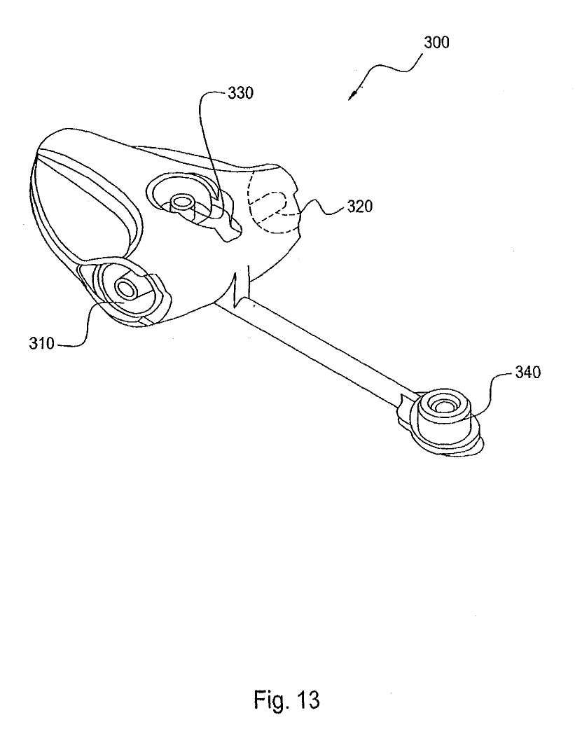

Referring to FIG. 13, T-connector 300 is a triangular shaped valve that allows a user to utilize either a single breast cup 400 or two breast cups through use of a first orifice 310 and a second orifice 320. Breast pump 100 is connected to t-connector 300 through air tubing 350 at inlet 330. The single split valve configuration of t-connector 300 minimizes the amount of tubing 350 necessary for double pumping. T-connector 300 has a plug 340 for closing off either of first or second orifices 310, 320 if single pumping is desired. Preferably, plug 340 is tethered to an outer surface of t-connector 300 to facilitate engagement with first or second orifices 310, 320.

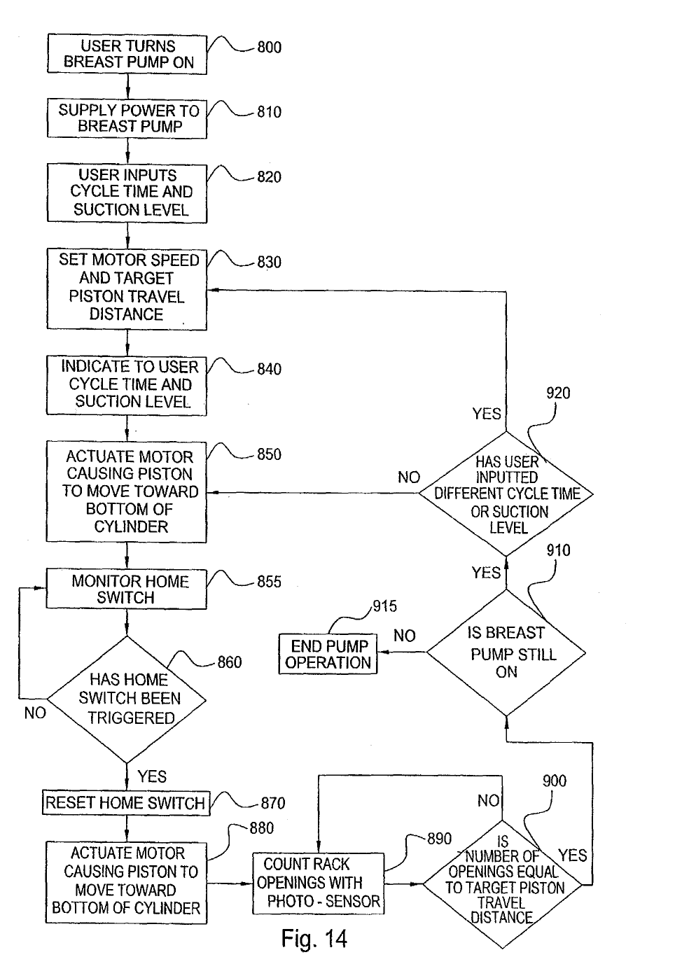

Referring to FIG. 14, a method of expressing breast milk according to the breast pump system of the present invention, is shown. The user commences the breast pumping operation by turning breast pump 100 “on,” as in step 800. This causes power to be supplied to breast pump 100 (step 810). The user then inputs the cycle time and suction level that is desired, as in step 820. In the preferred embodiment, the user has five cycle times and suction levels from which to choose. The cycle time and suction level is inputted by use of button pad 105.

In step 830, PC board 120 sets the motor speed and target piston travel distance according to the user's inputted levels for cycle time and suction. The cycle time and suction level are then displayed to the user, as in step 840. In this embodiment, the cycle time and suction level are indicated by lights 225 with the number of illuminated lights corresponding to the level. In step 850, motor 125 is actuated causing piston 112 to move toward bottom 175 of cylinder 113. This creates a positive pressure that is supplied to breast cup 400 by air tubing 350.

In step 855, the PC Board monitors the home switch to determine whether it has been triggered by contact with piston 112. In step 860, it is determined whether the home switch has been triggered. If the home switch has been triggered then it is reset as in step 870. In step 880, motor 125 is then reversed causing piston 112 to move toward top 180 of cylinder 113. This creates a negative pressure that is supplied to breast cup 400 by air tubing 350. One of the advantages of the breast pump system of the present invention is that is supplies both a positive pressure and a negative pressure through the same air tubing 350. This reduces cleaning and simplifies the operation for a user.

To provide the proper amount of suction as inputted by the user, photo-sensors 121 count the number of rack openings 50, as in step 890. In step 900, PC board 120 determines if the number of rack openings 50 that have been counted is the equivalent of the target piston travel distance as inputted by the user. In step 910, it is determined whether breast pump 100 is still “on.” If breast pump 100 has been shut off then the pumping operation ends, as in step 915.

In step 920, it is determined whether the user has inputted a new cycle time or suction level. If a new cycle time or suction level has been inputted, then PC Board 120 sets the motor speed and target piston travel distance according to the user's inputted levels for cycle time and suction, reverting back to step 830 and repeating the above described steps. If the user has not inputted a new cycle time or suction level then the motor is again reversed causing piston 112 to move toward bottom 175 of cylinder 113. This creates a positive pressure that is supplied to breast cup 400 by air tubing 350. The process continues with breast pump 100 supplying positive pressure and then negative pressure to breast cup 400 until the breast pump is shut off (step 910).

The breast pump system of the present invention includes a number of components and can be used in remote locations, such as when a user is traveling. The various components can be disposed within a bag system for ease of use. An example of such a bag system, as well as the components of such a system, is disclosed in the co-pending and commonly owned U.S. application Ser. No. 10/331,130, filed Dec. 27, 2002, the disclosure of which is incorporated herein by reference.

The present invention having been thus described with particular reference to the preferred forms thereof, it will be obvious that various changes and modifications may be made therein without departing from the spirit and scope of the present invention as defined in the appended claims.

What is claimed is:

1. A breast pump system for expressing breast milk from a breast, the system comprising:
a pressure source for generating a positive pressure and a negative pressure; and
a breast cup in fluid communication with said pressure source, wherein said breast cup applies said positive pressure and said negative pressure to said breast.

2. The system of claim 1, further comprising a channel, wherein said breast cup has an air orifice, wherein said channel is connected to said air orifice and said pressure source, and wherein said pressure source supplies reciprocating air flow through said channel between said breast cup and said pressure source.

3. The system of claim 2, wherein said channel is flexible tubing.

4. The system of claim 2, wherein said pressure source is a piston movably disposed in a cylinder.

5. The system of claim 4, further comprising a motor, a rack having first teeth and a gear having second teeth, wherein said rack is connected to said piston, said gear is operably connected to said motor, and said first teeth engage with said second teeth to reciprocally move said piston in said cylinder.

6. The system of claim 4, wherein said piston has a sealing member disposed between said piston and said cylinder.

7. The system of claim 6, wherein said sealing member is an o-ring disposed on said piston.

8. The system of claim 6, wherein said piston has a substantially cylindrical shape with a circumferential wall, and said sealing member is a plurality of gaskets disposed on said circumferential wall.

9. The system of claim 6, wherein said piston has a substantially cylindrical shape with a circumferential wall having a circumferential channel formed therein, and wherein said sealing member is at least partially disposed in said channel.

10. The system of claim 4, wherein said piston has a v-shaped cross section with a leading edge and a trailing edge, and wherein said leading edge and said trailing edge form a sealing engagement with said cylinder.

11. The system of claim 5, wherein said piston is flexibly secured to said rack.

12. The system of claim 11, wherein said piston has a recess and said rack has a first end with an abutment formed therein, and wherein said abutment is flexibly secured in said recess.

13. The system of claim 12, wherein said recess and said first end have detent structures.

14. The system of claim 5, wherein said cylinder has a first diameter and an air hole, wherein said air hole has a second diameter and is in fluid communication with said atmosphere, and wherein said first diameter is significantly larger than said second diameter.

15. The system of claim 5, further comprising a controller operably connected to said motor, wherein said motor is reversible and said controller reverses said motor based upon a positive or negative pressure limit.

16. The system of claim 5, further comprising a controller operably connected to said motor, wherein said motor is a reversible motor, wherein said controller determines a distance that said piston has traveled relative to said cylinder and wherein said controller reverses said motor based upon said distance.

17. The system of claim 16, further comprising a photo-sensor that generates a signal in response to said distance, wherein said signal is transmitted to said controller, and wherein said controller reverses said motor in response to said signal.

18. The system of claim 17, wherein said rack has a plurality of openings formed therein, wherein said photo-sensor is operably aligned with said openings, and wherein said signal is generated based upon a count of said openings moving past said photo-sensor.

19. The system of claim 18, further comprising a position switch, wherein said photo-sensor is operably aligned with said position switch to generate a position signal, wherein said position signal is transmitted to said controller, and wherein said controller resets said count in response to said position signal.

20. The system of claim 5, further comprising a controller operably connected to said motor, wherein said motor has variable speed, and said controller adjusts said speed based upon a desired cycle time for applying said positive or negative pressure to said breast.

21. The system of claim 20, wherein said controller has a user interface, said desired cycle time is inputted into said user interface, and said desired cycle time is transmitted to said controller from said user interface.

22. The system of claim 1, further comprising a controller having a user interface and operably connected to said pressure source, wherein said controller adjusts the positive or negative pressure generated by the pressure source in response to a signal transmitted from said user interface.

23. The system of claim 1, further comprising a controller having a user interface and operably connected to said pressure source, wherein said controller adjusts a cycle time for applying said positive or negative pressure to said breast in response to a signal transmitted to said controller from said user interface.

24. The system of claim 5, further comprising a controller that generates a wave signal in response to an amount of pressure and a cycle time between said positive and negative pressure, and controls said motor in response to said wave signal.

25. The system of claim 24, further comprising a user interface, wherein a desired wave signal is inputted into said user interface, said desired wave signal is transmitted to said controller from said user interface, and said controller adjusts said wave signal to correspond to said desired wave signal.

26. The system of claim 4, wherein said cylinder is in fluid communication with a pressure relief valve.

27. The system of claim 26, wherein said pressure relief valve is adjustable.

28. The system of claim 3, wherein said pressure source has a housing with a storage compartment formed therein, and wherein said flexible tubing is removably stored in said storage compartment.

29. The system of claim 28, wherein said housing has an air outlet with a first end and a second end, wherein said first end is in fluid communication with said pressure source and said second end is disposed in said storage compartment.

30. The system of claim 1, further comprising a t-connector having an inlet, a first outlet, a second outlet and a plug, wherein said inlet is in fluid communication with said first and second outlets, and wherein said plug is selectively sealingly engageable with said first outlet or said second outlet.

31. The system of claim 30, wherein said t-connector has an outer surface and said plug is tethered to said outer surface.

32. A breast pump system for expressing breast milk from a breast, the system comprising:
a cylinder having a cylinder volume;
a piston movably disposed in said cylinder;
a motor operably connected to said piston to generate a pressure in said cylinder volume; and
a breast cup in fluid communication with said cylinder volume, wherein said breast cup applies said pressure to said breast.

33. The system of claim 32, further comprising a channel, wherein said breast cup has an air orifice, wherein said channel is connected to said air orifice and said cylinder volume, and wherein said cylinder volume supplies reciprocating air flow through said channel.

34. The system of claim 33, wherein said channel is flexible tubing.

35. The system of claim 32, further comprising a rack having first teeth and a gear having second teeth, wherein said rack is connected to said piston, said gear is operably connected to said motor, and said first teeth engage with said second teeth to reciprocally move said piston in said cylinder.

36. The system of claim 32, wherein said piston has a sealing member disposed between said piston and said cylinder.

37. The system of claim 36, wherein said sealing member is an o-ring disposed on said piston.

38. The system of claim 36, wherein said piston has a substantially cylindrical shape with a circumferential wall, and said sealing member is a plurality of gaskets disposed on said circumferential wall.

39. The system of claim 36, wherein said piston has a substantially cylindrical shape with a circumferential wall having a circumferential channel formed therein, and wherein said sealing member is at least partially disposed in said channel.

40. The system of claim 32, wherein said piston has a v-shaped cross section with a leading edge and a trailing edge, and wherein said leading edge and said trailing edge form a sealing engagement with said cylinder.

41. The system of claim 35, wherein said piston is flexibly secured to said rack.

42. The system of claim 41, wherein said piston has a recess and said rack has a first end with an abutment formed therein, and wherein said abutment is flexibly secured in said recess.

43. The system of claim 42, wherein said recess and said first end have detent structures.

44. The system of claim 32, wherein said cylinder has a first diameter and an air hole, said air hole has a second diameter and is in fluid communication with said atmosphere, and said first diameter is significantly larger than said second diameter.

45. The system of claim 32, further comprising a controller operably connected to said motor, wherein said motor is reversible and said controller reverses said motor based upon a pressure limit.

46. The system of claim 35, further comprising a controller operably connected to said motor, wherein said motor is reversible, wherein said controller determines a distance that said piston has traveled relative to said cylinder and wherein said controller reverses said motor based upon said distance.

47. The system of claim 46, further comprising a photo-sensor that generates a signal in response to said distance, wherein said signal is transmitted to said controller.

48. The system of claim 47, wherein said rack has a plurality of openings formed therein, wherein said photo-sensor is operably aligned with said openings, and wherein said signal is generated based upon a count of said openings moving past said photo-sensor.

49. The system of claim 48, further comprising a position switch, wherein said photo-sensor is operably aligned with said position switch to generate a position signal, wherein said position signal is transmitted to said controller, and wherein said controller resets said count in response to said position signal.

50. The system of claim 32, further comprising a controller operably connected to said motor, wherein said motor is variable speed and said controller adjusts said speed based upon a desired cycle time for applying said pressure to said breast.

51. The system of claim 50, wherein said controller has a user interface, said desired cycle is inputted into said user interface, and said desired cycle time is transmitted to said controller from said user interface.

52. The system of claim 32, further comprising a controller having a user interface and operably connected to said motor, wherein said controller adjusts the pressure generated in said cylinder volume in response to a signal transmitted from said user interface.

53. The system of claim 32, further comprising a controller having a user interface and operably connected to said motor, wherein said controller adjusts a desired cycle time for applying said pressure to said breast in response to a signal transmitted to said controller from said user interface.

54. The system of claim 32, further comprising a controller that generates a wave signal in response to an amount of pressure and a cycle time for said pressure, and controls said motor in response to said wave signal.

55. The system of claim 54, further comprising a user interface, wherein a desired wave signal is inputted into said user interface, said desired wave signal is transmitted to said controller from said user interface, and wherein said controller adjusts said wave signal to correspond to said desired wave signal.

56. The system of claim 32, wherein said cylinder is in fluid communication with a pressure relief valve.

57. The system of claim 56, wherein said pressure relief valve is adjustable.

58. The system of claim 34, further comprising a housing having a storage compartment, wherein said motor, said piston and said cylinder are disposed in said housing, and wherein said flexible tubing is removably stored in said storage compartment.

59. The system of claim 58, wherein said housing has an air outlet with a first end and a second end, wherein said first end is in fluid communication with said cylinder volume and said second end is disposed in said storage compartment.

60. The system of claim 32, further comprising a t-connector having an inlet, a first outlet, a second outlet and a plug, wherein said inlet is in fluid communication with said first and second outlets, and wherein said plug is selectively sealingly engageable with said first outlet or said second outlet.

61. The system of claim 60, wherein said t-connector has an outer surface and said plug is tethered to said outer surface.

62. A breast pump for expressing breast milk from a breast, the pump comprising:
a pressure source for generating a positive pressure and a negative pressure; and
a controller operably connected to the pressure source, wherein said controller adjusts said positive and negative pressure and adjusts a cycle time between application of said positive and negative pressure to said breast.

63. The pump of claim 62, wherein said controller has a user interface, a desired cycle time is inputted into said user interface, said desired cycle time is transmitted to said controller from said user interface, and said controller adjusts said cycle time to correspond to said desired cycle time.

64. The pump of claim 62, wherein said controller has a user interface, a desired level of said positive or negative pressure is inputted into said user interface, and said controller adjusts said positive or negative pressure in response to a signal transmitted from said user interface.

65. The pump of claim 62, wherein said controller generates a wave signal in response to said pressure and said cycle time, and controls said pressure source in response to said wave signal.

66. The pump of claim 65, further comprising a user interface, wherein a desired wave signal is inputted into said user interface, said desired wave signal is transmitted to said controller from said user interface, and said controller adjusts said wave signal to correspond to said desired wave signal.

67. A breast pump for expressing breast milk from a breast, the pump comprising:
a pressure source for generating a pressure; and
a controller operably connected to the pressure source, wherein said controller adjusts said pressure and adjusts a cycle time between application of said pressure to said breast, and wherein said controller generates a wave signal in response to said pressure and said cycle time, and controls said pressure source in response to said wave signal.

68. The pump of claim 67, further comprising a user interface, wherein a desired wave signal is inputted into said user interface, said desired wave signal is transmitted to said controller from said user interface, and said controller adjusts said wave signal to correspond to said desired wave signal.

69. A drive system for an expandable volume of a breast pump, the system comprising:
a motor having a first rotary output;
a first gear system operably connected to said motor; and
a second gear system operably connected to said first gear system and the expandable volume, said second gear system having a rack gear and a pinion gear operably connected to said rack gear,
wherein said first rotary output is provided to said second gear system,
wherein said first gear system adjusts said first rotary output provided to said second gear system to a second rotary output, and
wherein said second gear system translates said second rotary output to a linear output.

70. The drive system of claim 69, wherein said first gear system has at least one belt.

71. The drive system of claim 70, wherein said at least one belt is a first belt and a second belt.

72. The drive system of claim 71, wherein said first belt is non-toothed and said second belt is toothed.

73. The drive system of claim 72, wherein said first belt is resilient.

74. The drive system of claim 73, wherein said first belt is a plurality of belts.

75. The drive system of claim 74, wherein said motor has a drive shaft with an annular channel formed therein, and wherein said first belt is partially disposed in said annular channel.

76. The drive system of claim 72, wherein said first gear system further comprises a first pulley and a second pulley, said first pulley having a first circumference and a first channel formed along said first circumference, said second pulley having a second circumference and a plurality of teeth formed along said second circumference, wherein said first belt is partially disposed in said first channel, and wherein said second belt engages said plurality of teeth of said second pulley.

77. The drive system of claim 76, wherein said second pulley is secured to said pinion gear.

78. A breast pump for supplying a pressure to a breast cup, said pump comprising:
an expandable volume in fluid communication with said breast cup to supply said pressure;
a motor having a first rotary output;
a first gear system operably connected to said motor; and
a second gear system operably connected to said first gear system and said expandable volume, said second gear system having a rack gear and a pinion gear operably connected to said rack gear,
wherein said first rotary output is provided to said second gear system,
wherein said first gear system adjusts said first rotary output provided to said second gear system to a second rotary output, and
wherein said second gear system translates said second rotary output to a linear output.

79. The drive system of claim 78, wherein said first gear system has at least one belt.

80. The drive system of claim 79, wherein said at least one belt is a first belt and a second belt.

81. The drive system of claim 80, wherein said first belt is non-toothed and said second belt is toothed.

82. The drive system of claim 81, wherein said first belt is resilient.

83. The drive system of claim 81, wherein said first belt is a plurality of belts.

84. The drive system of claim 83, wherein said motor has a drive shaft with an annular channel formed therein, and wherein said first belt is partially disposed in said annular channel.

85. The drive system of claim 81, wherein said first gear system further comprises a first pulley and a second pulley, said first pulley having a first circumference and a first channel formed along said first circumference, said second pulley having a second circumference and a plurality of teeth formed along said second circumference, wherein said first belt is partially disposed in said first channel, and wherein said second belt engages said plurality of teeth of said second pulley.

86. The drive system of claim 85, wherein said second pulley is secured to said pinion gear.

87. The breast pump of claim 78, wherein said expandable volume comprises a cylinder and a piston movable in said cylinder, and wherein said rack gear is secured to said piston.

88. The breast pump of claim 87, wherein said rack gear is flexibly secured to said piston.

89. The breast pump of claim 87, wherein said cylinder has a first diameter and an air hole, said air hole has a second diameter and is in fluid communication with said atmosphere, and said first diameter is significantly larger than said second diameter.

90. The breast pump of claim 87, further comprising a controller operably connected to said motor, wherein said motor is reversible, wherein said controller determines a distance that said piston has traveled relative to said cylinder, and wherein said controller reverses said motor based upon said distance.

91. The breast pump of claim 78, further comprising a controller operably connected to said motor, wherein said motor is reversible and said controller reverses said motor based upon a pressure limit.

92. The breast pump of claim 78, further comprising a controller operably connected to said motor, wherein said motor is variable speed and said controller adjusts said speed based upon a desired cycle time for supplying said pressure to said breast cup.
